(19) United States
(12) Patent Application Publication
(10) Pub. No.: US 2023/0263941 A1
(43) Pub. Date: Aug. 24, 2023

(54) SYSTEMS, METHODS, AND DEVICES INCLUDING A DEMINERALIZED BONE MATRIX (DBM) GRAFT WITH GELATIN CARRIER

(71) Applicant: ThoraGenix Innovations, Inc., Boca Raton, FL (US)

(72) Inventor: Fotios M. ANDREOPOULOS, Miami Shores, FL (US)

(21) Appl. No.: 18/174,327

(22) Filed: Feb. 24, 2023

Related U.S. Application Data
(60) Provisional application No. 63/313,376, filed on Feb. 24, 2022.

(51) Int. Cl. A61L 27/36; A61L 27/44; A61L 27/54; A61L 27/38

(52) U.S. Cl. CPC A61L 27/3608; A61L 27/365; A61L 27/3687; A61L 27/3691; A61L 27/3821; A61L 27/44; A61L 27/54; A61L 2300/402; A61L 2300/406; A61L 2300/418

(57) ABSTRACT

Systems, methods, and devices include techniques for generating and using a demineralized bone matrix (DBM)-gelatin matrix allograft material. The DBM-gelatin material can be used to form an implant (e.g., for sternal closure operations) and/or a gel (e.g., for wound/fracture treatment). A method for forming the implant or bone graft can include forming the DBM from an initial bone material; and mixing, in a solution, the DBM with a gelatin carrier to form a DBM-gelatin solution. The gelatin carrier can include an animal-based collagen, such as a porcine-based collagen or a bovine-based collagen. Additionally, the method of forming the bone graft can include performing a crosslinking reaction with the DBM-gelatin solution. The implant can be packaged in a sterile hydration container prior to use.

### CROSS-REFERENCE TO RELATED APPLICATIONS

This application claims priority to U.S. Provisional Pat. Application No. 63/313,376, filed Feb. 24, 2022, and titled “SYSTEMS, METHODS, AND DEVICES INCLUDING A DEMINERALIZED BONE MATRIX (DBM) GRAFT WITH GELATIN CARRIER,” which is incorporated by reference in its entirety.

### BACKGROUND

Wounds and bone fractures result in millions of medical operations, globally each year. Bone fracture sites are highly prone to infections. Some types of bone infections, such as sternal infections, can have mortality rates upwards of fifty percent. Furthermore, various types of tissue wounds, such as gunshots or knife wounds, can result in dangerous infections or blood loss if not treated immediately and effectively.

It is with these observations in mind, among others, that various aspects of the present disclosure were conceived and developed.

### BRIEF SUMMARY

The systems, methods, and devices disclosed herein can address the aforementioned problems by providing a demineralized bone matrix (DBM) graft with a gelatin carrier. For instance a method for forming a bone graft can include forming the DBM from an initial bone material; mixing, in a solution, the DBM with a gelatin carrier to form a DBM-gelatin solution; and/or performing a crosslinking reaction with the DBM-gelatin solution to form a DMB-gelatin matrix allograft material.

In some examples, forming the DBM from the initial bone material includes milling or grinding cut bone pieces to form a bone powder having 200-1000 µm particles, and/or sieving the bone powder for 400-800 µm particles. Furthermore, forming the DBM from the initial bone material can include demineralizing a bone powder via three incubations of one hour with 0.5-N HCl to create an initial DBM material. Forming the DBM from the initial bone material can also include washing the initial DBM material with water until reaching a pH of 7.4 to form washed DBM material; freezing the washed DBM material at -80° C. for 48 hours to form frozen DBM material; and/or lyophilizing the frozen DBM material for 48 hours.

In some examples, the DBM is mixed with the gelatin carrier to form the DBM-gelatin solution at a DBM:gelatin carrier ratio of 20% to 50% w/w%. The gelatin carrier can be a porcine gelatin. Moreover, performing the crosslinking reaction can include adding, to the DBM-gelatin solution, a solution of N-hydroxysuccinimide/1-ethyl-3-(3-(dimethylaminopropyl)carbodiimide hydrochloride (NHS/EDC). The solution of NHS/EDC can be added to the DBM-gelatin solution at a NHS/EDC:DBM-gelatin solution ratio of between 1:10 and 5:10. Furthermore, the crosslinking reaction can be performed in a cast to form a DBM-gelatin allograft implant with a shape predetermined by the cast. The method can also include packaging the DBM-gelatin allograft implant in a sterile hydration container. Additionally or alternatively, the method can include securing the DBM-gelatin allograft implant between two portions of severed bone to promote bone growth between the two portions of severed bone (e.g., for a sternal closure procedure).

In some examples, a method for forming a bone graft includes providing DBM formed from an initial bone material; mixing the DBM with a collagen-based gelatin carrier to form a DBM-gelatin solution; and/or performing a crosslinking reaction with the DBM-gelatin solution to form a DBM-gelatin matrix allograft material. The method can also include drying the DBM-gelatin matrix allograft material a period of time to form a firm implant having a low-moisture collagen carrier; and/or packaging the firm implant in a saline hydration container. Moreover, the method can include drying the DBM-gelatin matrix allograft material a period of time to form a gel having a high-moisture collagen carrier; and/or applying the gel to a wound treatment device. Additionally or alternatively, the high-moisture collagen carrier can include at least one of an antibiotic, a blood clotting agent, an anesthetic, or a bone growth cell. Also the collagen-based gelatin carrier can include a porcine-based collagen, a bovine-based collagen, a human-based collagen, or a plant-based collagen.

In some instances, a method for forming a bone graft includes forming a demineralized bone matrix (DBM) from a bone powder having 400-800 µm particles; mixing the DBM with a gelatin carrier, which is porcine collagen-based or bovine collagen-based, to form a DBM-gelatin solution; and/or performing a crosslinking reaction with the DBM-gelatin solution to form a DBM-gelatin matrix allograft. Additionally, the method can includes drying the DBM-gelatin matrix allograft a predetermined time period to reach a predefined moisture content, the predefined moisture content corresponding to a type of allograft. In some scenarios, the type of allograft is at least one of a gel or a collagen-based implant.

The foregoing is intended to be illustrative and is not meant in a limiting sense. Many features of the examples may be employed with or without reference to other features of any of the examples. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the disclosed subject matter. It should be understood, however, that the disclosed subject matter is not limited to the precise examples and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including a Demineralized Bone Matrix (DBM) gelatin carrier graft, as discussed in greater detail below;

FIG. 2 illustrates an example method for generating a DBM gelatin carrier graft, which can form at least a portion of the system depicted in FIG. 1; and

FIG. 3 illustrates an example method for forming the DBM material of a DBM gelatin carrier graft, which can form at least a portion of the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Further, as the presently disclosed technology is susceptible to examples of many different forms, it is intended that the present disclosure be considered as an example of the principles of the presently disclosed technology and not intended to limit the presently disclosed technology to the specific examples shown and described. Any one of the features of the presently disclosed technology may be used separately or in combination with any other feature. References to the terms “example,” “examples,” and/or the like in the description mean that the feature and/or features being referred to are included in, at least, one aspect of the description. Separate references to the terms “example,” “examples,” and/or the like in the description do not necessarily refer to the same example and are also not mutually exclusive unless so stated and/or except as will be readily apparent to those skilled in the art from the description. For example, a feature, structure, process, step, action, or the like described in one example may also be included in other examples, but is not necessarily included. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

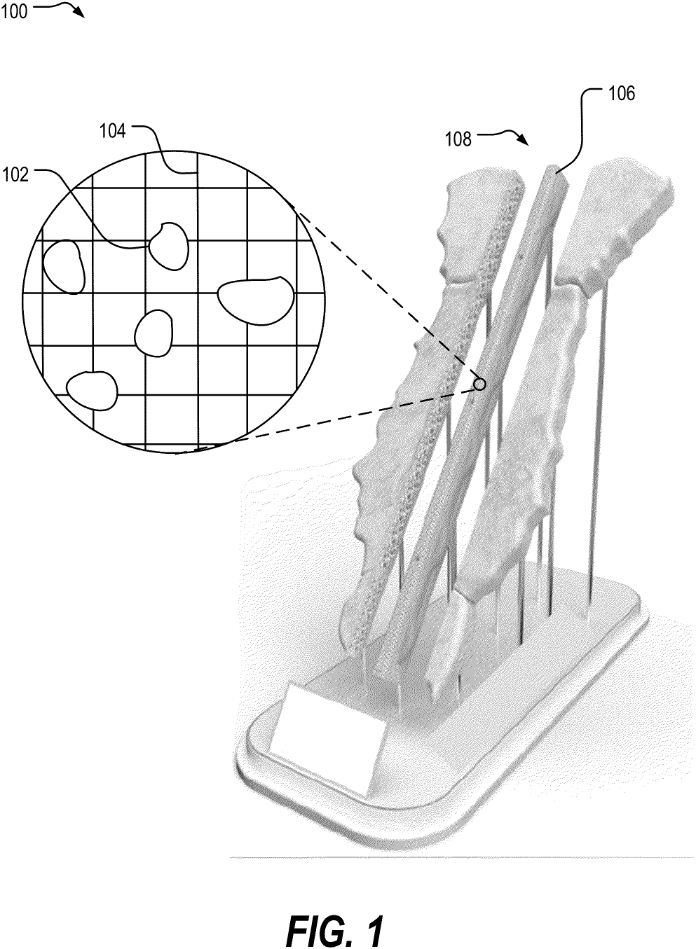

FIG. 1 illustrates an example system 100 including a demineralized bone matrix (DBM) 102 with a gelatin carrier 104.

In some scenarios, the DBM 102 can be harvested from cadaveric human bone according to tissue procurement guidelines advocated by the American Association of Tissue Banks (AATB) with respect to donor screening and sterilization to minimize any disease transmission between the donor and the patient. The bone source for DBM production can be the femur diaphysis, the pelvic bone (ilium), and/or any other type of bone.

Furthermore, the bone pieces that form the DBM 102 can be denuded from soft tissue and cut with a circular saw to smaller pieces, and then milled using either Universal Bone Mill (e.g., from Cenna Medical Technologies) or other similar mills. The Universal Bone Mill offers the possibility to re-mill the initially milled pieces for improved control of the bone fragment size and distribution. Additionally or alternatively, a bone grinder could be used to create the bone powder in much smaller sizes (e.g., 200-1000 µm). An electromagnetic sieve shaker could then be used to further control the size distribution of the bone particles. For instance, a bone particle sizes ranging between 400 and 800 micrometers can be used, which can result in improved osteoconductivity. The cortical bone powder can then be washed and defatted through a plurality of sequential washes (e.g., three washes) with milliQ water (e.g., from Millipore Corporation, MA, USA) at 60° C., with 70% ethanol (e.g., from Sigma-Aldrich, St. Louis, MO, USA), and then absolute ethanol (e.g., Sigma-Aldrich). Following the washing process the bone powder can dry at room temperature overnight. The cortical powder can then be demineralized through one or more (e.g., three) incubations of 1 hour with 0.5-N HCI (Sigma-Aldrich), followed by washing with milliQ water until reaching a neutral pH of 7.4. The DBM samples can then be frozen at -80° C. for 48 hours, lyophilized for 48 hours, and stored at -80° C. until use. This demineralization procedure can take place inside a laminar flow hood under aseptic conditions. In some scenarios, however, DBM fiber bone can be used rather than DBM powder.

In some examples, gelatin 104 can be used as a carrier with the DBM 102. Porcine Gelatin (e.g., GA; G1890, Bloom 300, porcine skin-type A, Sigma Aldrich) can be used as the binding carrier. Other types of animal-based collagens can be used additionally or alternatively to porcine, such as bovine collagen, human collagen, and/or plant collagen. An EDC/NHS coupling chemistry can be used to crosslink the gelatin carrier 104 and DBM 102. DBM 102 can be mixed with gelatin powder in phosphate-buffered saline (PBS) at ratios ranging from 20% to 50 w/w% with respect to the carrier (DBM:Gelatin). A gelatin solution can be prepared at 10 w/v% in PBS (e.g., 0.1 g/ml) and the gelatin can completely dissolve under light heating (e.g., 37° C.) and stirring. Once the gelatin solution is homogeneous, an appropriate amount of DBM 102 (20-50 w/w% ratios) can be added to the gelatin solution. A solution of a cross-link activator, such as NHS/EDC (N-hydroxysuccinimide) / (1-ethyl-3-(3-(dimethylaminopropyl) carbodiimide hydrochloride) can be prepared by mixing 0.4 mg of EDC and 0.6 mg of NHS into a 1 ml of PBS. 1 ml of the NHS and EDC solution can be added to the DBM/gelatin solution (e.g., 10 ml) to initiate a crosslinking reaction. The amount of EDC/NHS added to the DBM/gelatin solution (e.g., 1 ml of EDC/NHS:10 ml of DBM/gelatin) can be increased to 2 ml:10 ml and 5 ml:10 ml respectively in order to adjust the crosslinking rate and subsequently the properties of the crosslinked matrix. EDC/NHS crosslinking can occur via the formation of stable amide bonds between the amines and the carboxylated groups of the gelatin. Two hours can elapse for the crosslinking to be completed. Once again, the crosslinker ratios and crosslinking time can be varied to manipulate the mechanical properties of the final DBM graft.

The crosslinking operation can be performed in a cast such that the formation of a gelatin/DBM matrix graft 106 with the appropriated dimensions required for the clinic (e.g. thickness, length and width) is created. Following crosslinking, the gelatin/DBM matrix graft 106 can be removed from the cast, incubated in milliQ water for 1 hour to remove any unreacted material, and placed in a lyophilizer to remove all solvents. Labconco offers a number of bench top lyophilizers (e.g. FreeZone 8 Liter -50° C. Benchtop Freeze Dryers).

In some examples, the cross-link activator ratio, the lyophilizer period, and/or a drying period for the gelatin carrier 104 can be set or adjusted to correspond to a particular characteristic of the gelatin carrier 104. For instance, a moisture content of the gelatin carrier can be controlled and adjusted between a high moisture content (e.g., for liquid, gel, and/or softer applications) and a low moisture content (e.g., for firm, hard, solid, rigid, and/or semi-rigid applications).

The lyophilized DBM/gelatin matrix graft 106 can then be sterilized by ethylene oxide according to standard, medical grade gas sterilizations techniques.

The techniques disclosed herein can provide an initial formulation of a DBM/gelatin matrix graft 106 suitable for in vitro and in vivo usage. The biological activity (e.g., osteoconductivity and osteoinductivity) of the DBM/gelatin matrix graft 106 can primarily be based on its chemical composition in relation to its protein content (e.g., collagens, growth factors, incorporation of bone morphogenic proteins), and/or the presence of residual calcium. A series of in vitro testing (i.e. cytotoxicity, alkaline phosphatase, modulus of elasticity) and in vivo assessment (i.e. bone formation, scarring, etc.) can be used to evaluate the graft performance and modulated critical parameters (e.g., carrier concentrations, particle size, and incorporation of biological additives).

Furthermore, the DBM/gelatin matrix graft 106 can form an allograft implant 108 (e.g., a rigid or semi-rigid implant) for homologous use for the repair, replacement or reconstruction of skeletal defects by a qualified healthcare professional (e.g., physician). The allografts can be manufactured using cadaveric bone, demineralized bone fiber, a collagen carrier, and/or potential non-cellular growth factors, e.g., exosome protein as well as BMP proteins. The allograft implant 108 can be free of viable organisms, and/or can be biocompatible.

The allograft 108 can be packaged in a pouch, a carton, in other accessories, etc. In one example, the packaging configuration is a sterile hydration carton. In some instances, the allograft implant 108 can have 24 months of shelf life, and/or real time aging can continue to extend shelf life for up to 5 years. The real time aging points can be 3, 6, 12, 18, 24 months then 3, 4, 5 years. Furthermore, the allograft implant 108 can be stored at room temperature. The allograft implant 108 can meet FDA and AATB standards, and/or can have a 361 HCT/P or 510(k) or PMA anticipated designation. In some instances, the allograft implant 108 can include a package insert with instructions for use. A label for the allograft implant 108 can contain at least the following: a product name, a part number, a lot number, a serial number, an expiration date, a manufacturer name, a distributor name, and/or a sterilization method (e.g., sterile filtration and irradiation). Additionally or alternatively, an RTI Tissue Utilization Record cab be included in the implant packaging.

Furthermore, in some examples, the gelatin carrier 104 can include a carried additive. For instance, additionally or alternatively to including the DBM 102, the gelatin carrier 104 can include one or more of an antibiotics, a blood clotting agent (e.g., thrombin), an anesthetic (e.g., ketamine, lidocaine, or so forth), and/or a bone growth cell (e.g., an exosome, a bone morphogenetic protein, a live stem growth cell).

In some examples, the manufacturing parameters can be modified to adjust a moisture content of the gelatin carrier 104 to create a final product with varying form factors, such as a liquid, a gel, or a putty. For instance, the gelatin carrier 104 can be a gel formed from one or more types of collagen, such as a porcine-based collagen, a bovine-based collagen, a human-based collagen, or a plant-based collagen, used to form a collagen-based carrier gel. The gelatin carrier gel can include the DBM 102 and/or any of the agents discussed herein (e.g., an antibiotics, a blood clotting agent, an anesthetic, and/or a bone growth cell. In some examples, the gelatin carrier gel can be a collagen matrix that absorbs into internal tissue of the patient. The collagen-based gelatin carrier can be applied to various wound treatment devices, such as bandages or internal wound treatment devices.

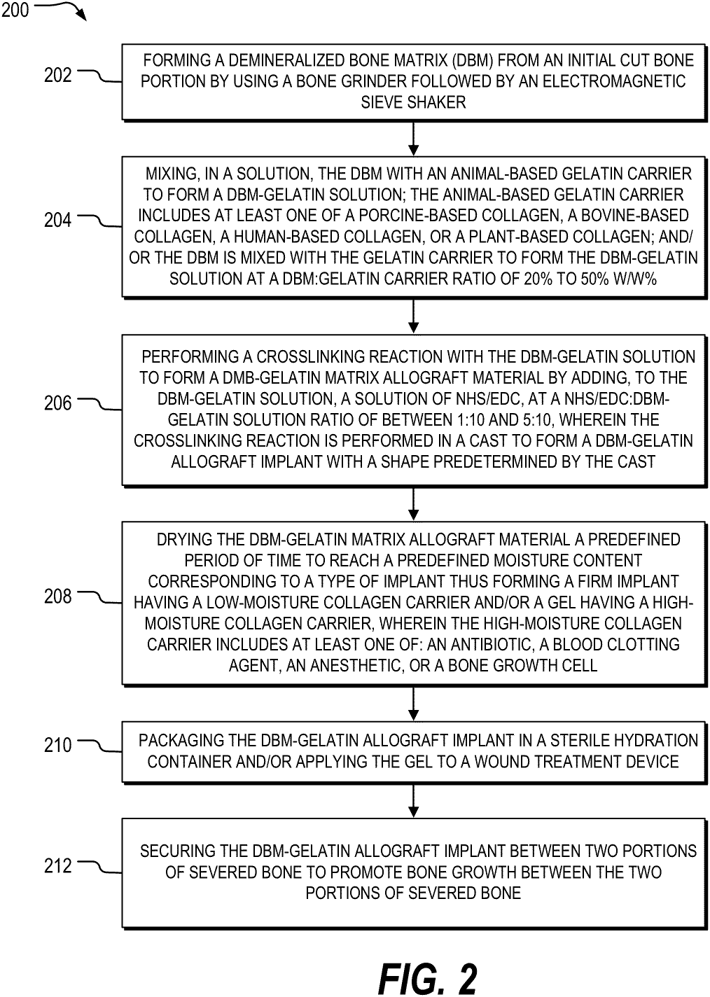

FIG. 2 illustrates an example method 200 of forming a DBM with a gelatin carrier, which can be combined with and/or used to form the system 100 discussed above regarding FIG. 1.

In some examples, at operation 202, the method 200 can form a DBM from an initial cut bone portion by using a bone grinder followed by an electromagnetic sieve shaker. At operation 204, the method 200 can mix, in a solution, the DBM with an animal-based gelatin carrier to form a DBM-gelatin solution; the animal-based gelatin carrier includes at least one of a porcine-based collagen, a bovine-based collagen, a human-based collagen, or a plant-based collagen; and/or the DBM can be mixed with the gelatin carrier to form the DBM-gelatin solution at a DBM:gelatin carrier ratio of 20% to 50% w/w%. At operation 206, the method 200 can perform a crosslinking reaction with the DBM-gelatin solution to form a DMB-gelatin matrix allograft material by adding, to the DBM-gelatin solution, a solution of NHS/EDC, at a NHS/EDC:DBM-gelatin solution ratio of between 1:10 and 5:10, wherein the crosslinking reaction can be performed in a cast to form a DBM-gelatin allograft implant with a shape predetermined by the cast. At operation 208 the method 200 can dry the DBM-gelatin matrix allograft material a predefined period of time to reach a predefined moisture content corresponding to a type of implant thus forming a firm implant having a low-moisture collagen carrier and/or a gel having a high-moisture collagen carrier, wherein the high-moisture collagen carrier includes at least one of: an antibiotic, a blood clotting agent, an anesthetic, or a bone growth cell. At operation 210, the method 200 can packaging the DBM-gelatin allograft implant in a sterile hydration container and/or applying the gel to a wound treatment device. At operation 212, the method 200 can secure the DBM-gelatin allograft implant between two portions of severed bone to promote bone growth between the two portions of severed bone.

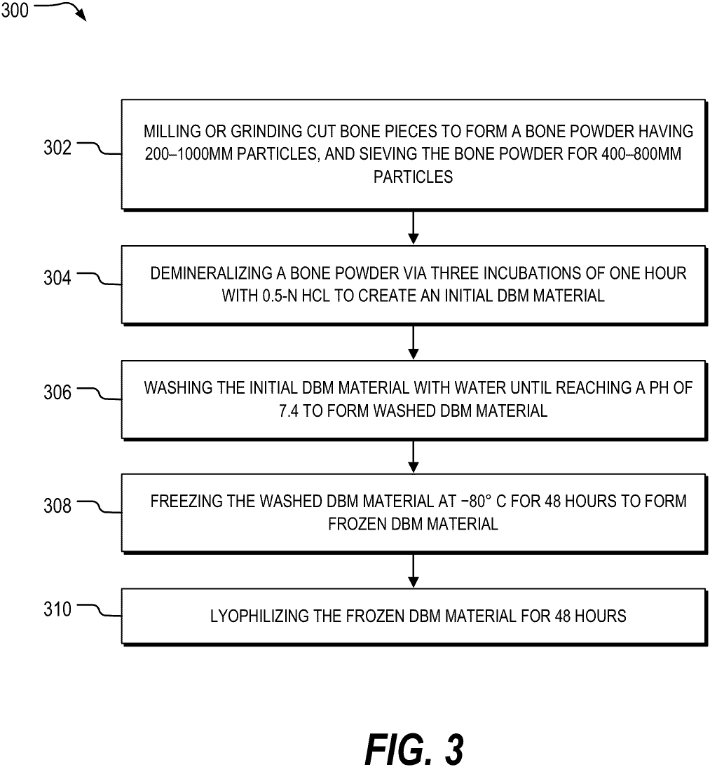

FIG. 3 illustrates an example method 300 of forming the DBM 102 to be used in a DBM gelatin matrix graft 106, which can be combined with and/or used to form the system 100 discussed above regarding FIG. 1.

In some examples, at operation 302, the method 300 can mill or grind cut bone pieces to form a bone powder having 200-1000 µm particles, and sieve the bone powder for 400-800 µm particles. At operation 304, the method 300 can demineralize a bone powder via three incubations of one hour with 0.5-N HCI to create an initial DBM material. At operation 306, the method 300 can wash the initial DBM material with water until reaching a pH of 7.4 to form washed DBM material. At operation 308, the method 300 can freeze the washed DBM material at -80° C. for 48 hours to form frozen DBM material. At operation 310, the method 300 can lyophilize the frozen DBM material for 48 hours.

It is to be understood that the specific order or hierarchy of steps in the methods discussed throughout this disclosure are instances of example approaches and can be rearranged while remaining within the disclosed subject matter. For instance, any of the operations discussed in FIGS. 2 and 3 and throughout this disclosure may be omitted, repeated, performed in parallel, performed in a different order, performed partially, and/or combined with any other of the operations or portions of the operations disclosed herein.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A method of forming a bone graft, the method comprising:
forming a demineralized bone matrix (DBM) from an initial bone material;
mixing, in a solution, the DBM with a gelatin carrier to form a DBM-gelatin solution; and
performing a crosslinking reaction with the DBM-gelatin solution to form a DMB-gelatin matrix allograft material.

2. The method of claim 1, wherein,
forming the DBM from the initial bone material includes milling or grinding cut bone pieces to form a bone powder having 200-1000 µm particles, and sieving the bone powder for 400-800 µm particles.

3. The method of claim 1, wherein,
forming the DBM from the initial bone material includes:
demineralizing a bone powder via three incubations of one hour with 0.5-N HCl to create an initial DBM material,
washing the initial DBM material with water until reaching a pH of 7.4 to form washed DBM material,
freezing the washed DBM material at -80° C. for 48 hours to form frozen DBM material, and
lyophilizing the frozen DBM material for 48 hours.

4. The method of claim 1, wherein,
the gelatin carrier includes a porcine-based collagen, a bovine-based collagen, a human-based collagen, or a plant-based collagen.

5. The method of claim 1, wherein,
the DBM is mixed with the gelatin carrier to form the DBM-gelatin solution at a DBM:gelatin carrier ratio of 20% to 50% w/w%.

6. The method of claim 1, wherein,
performing the crosslinking reaction includes adding, to the DBM-gelatin solution, a solution of N-hydroxysuccinimide/1-ethyl-3-(3-(dimethylaminopropyl)carbodiimide hydrochloride (NHS/EDC).

7. The method of claim 6, wherein,
the solution of NHS/EDC is added to the DBM-gelatin solution at a NHS/EDC:DBM-gelatin solution ratio of between 1:10 and 5:10.

8. The method of claim 1, wherein,
the crosslinking reaction is performed in a cast to form a DBM-gelatin allograft implant with a shape predetermined by the cast.

9. The method of claim 8, further including,
packaging the DBM-gelatin allograft implant in a sterile hydration container.

10. The method of claim 9, further including,
securing the DBM-gelatin allograft implant between two portions of severed bone to promote bone growth between the two portions of severed bone.

11. A method of forming a bone graft, the method comprising:
forming a demineralized bone matrix (DBM) from an initial cut bone portion by using a bone grinder followed by an electromagnetic sieve shaker;
mixing the DBM with an animal-based gelatin carrier to form a DBM-gelatin solution; and
performing a crosslinking reaction with the DBM-gelatin solution to form a DBM-gelatin matrix allograft material.

12. The method of claim 11, further including,
drying the DBM-gelatin matrix allograft material a period of time to form a firm implant having a low-moisture collagen carrier.

13. The method of claim 12, further including,
packaging the firm implant in a saline hydration container.

14. The method of claim 11, further including,
drying the DBM-gelatin matrix allograft material a period of time to form a gel having a high-moisture collagen carrier.

15. The method of claim 14, further including,
applying the gel to a wound treatment device.

16. The method of claim 14,
wherein the high-moisture collagen carrier includes at least one of:
an antibiotic,
a blood clotting agent,
an anesthetic, or
a bone growth cell.

17. The method of claim 11, wherein,
the animal-based gelatin carrier includes a porcine-based collagen or a bovine-based collagen.

18. A method of forming a bone graft, the method comprising:
forming a demineralized bone matrix (DBM) from a bone powder having 400-800 µm particles;
mixing the DBM with a gelatin carrier to form a DBM-gelatin solution, the gelatin carrier being porcine collagen-based or bovine collagen-based; and
performing a crosslinking reaction with the DBM-gelatin solution to form a DBM-gelatin matrix allograft.

19. The method of claim 18, further including:
drying the DBM-gelatin matrix allograft a predetermined time period to reach a predefined moisture content, the predefined moisture content corresponding to a type of allograft.

20. The method of claim 19, wherein,
the type of allograft is at least one of a gel or an implant.
